(19) United States
(12) Patent Application Publication
(10) Pub. No.: US 2025/0189645 A1
(43) Pub. Date: Jun. 12, 2025

(54) ULTRASONIC IMAGING SYSTEM AND METHOD FOR CONTROLLING SYNCHRONIZATION

(71) Applicant: FUJIFILM Corporation, Tokyo (JP)

(72) Inventors: Hirozumi TAKESHIMA, Tokyo (JP); Tomohiko TANAKA, Tokyo (JP)

(73) Assignee: FUJIFILM Corporation, Tokyo (JP)

(21) Appl. No.: 18/964,647

(22) Filed: Dec. 1, 2024

(30) Foreign Application Priority Data
Dec. 8, 2023 (JP) 2023-207925

(51) Int. Cl. G01S 7/52; G01S 15/89

(52) U.S. Cl. CPC G01S 7/52087; G01S 7/52004; G01S 15/8965

(57) ABSTRACT

An ultrasonic imaging system including an insertion member, a calculator, an estimator, and a synchronization controller are provided. The insertion member inserted into a living body has a light absorption element that absorbs a light pulse to generate a photoacoustic wave. The calculator calculates an actual measurement value of a synchronization deviation based on a reception signal sequence before a phase alignment addition or a pseudo-reception signal sequence corresponding to the reception signal sequence. The estimator estimates an estimation value of the synchronization deviation based on a synchronization deviation history. The synchronization controller changes a light pulse cycle or a reception cycle based on the actual measurement value in a case where the photoacoustic wave is detected, or changes the light pulse cycle or the reception cycle based on the estimation value in a case where the photoacoustic wave is not detected.

### CROSS-REFERENCE TO RELATED APPLICATION

This application claims priority under 35 USC 119 from Japanese Patent Application No. 2023-207925, filed Dec. 8, 2023, the disclosure of which is incorporated by reference herein.

### BACKGROUND OF THE INVENTION

### 1. Field of the Invention

The present disclosure relates to an ultrasonic imaging system and a method for controlling synchronization, and more particularly, relates to a technology for displaying a position of an insertion object inserted into a living body.

### 2. Description of the Related Art

An ultrasonic imaging system is a system for performing treatment or examination of a living body that is a patient, using an ultrasound imaging apparatus. More specifically, in the ultrasonic imaging system, an image representing a position of an insertion member inserted into a living body is generated and displayed. The treatment or the like of the living body is performed with reference to such an image. From such a viewpoint, the ultrasonic imaging system is a system that supports the treatment of the living body or the like.

In the ultrasonic imaging system, an ultrasound diagnostic apparatus is generally used as the ultrasound imaging apparatus. The insertion member inserted into the living body is, for example, a catheter inserted into a blood vessel. In general, a guide wire is inserted into the blood vessel before the insertion of the catheter. The guide wire is also an insertion member. An ultrasonic imaging method that images an insertion member inserted into a blood vessel using an ultrasound probe that comes into contact with a surface of a living body is also referred to as an extra-vascular ultrasound (EVUS).

An advanced ultrasonic imaging system that images an insertion member using a photoacoustic effect is proposed. In such an ultrasonic imaging system, a light absorption element is provided at a distal end of an insertion member. The light pulse generated by a light pulse generator is guided into an inside of the insertion object through an optical fiber, and the light absorption element is irradiated with the light pulse. Absorption of the light pulse by the light absorption element causes a photoacoustic wave to be generated in the living body. The photoacoustic wave is received by the ultrasound probe that comes into contact with the surface of the living body. Based on reception information obtained as described above, an image (hereinafter, referred to as a photoacoustic image or a PA image) representing a position of the light absorption element (that is, a sound source) is formed. For example, the photoacoustic image is composited with an ultrasound image (hereinafter, also referred to as a US image) generated by transmission and reception wave of an ultrasound wave. A composite image generated in this way is displayed. Through observation of the composite image, it is possible to clearly specify a position of the distal end of the insertion member while a biological tissue is observed.

In the above-described advanced ultrasonic imaging system, it is necessary to precisely synchronize a light pulse cycle in the light pulse generator and a reception cycle in the ultrasound probe. Even in a case where the sound source is located below the ultrasound probe, the sound source is not imaged as long as a timing at which the photoacoustic wave from the sound source reaches the ultrasound probe deviates from a reception period. In addition, in a case where the synchronization is not established, reception beam forming is not appropriately executed, and a quality of the image deteriorates. For the same reason, in a case where detection of the photoacoustic wave is interrupted due to some reason in a synchronization establishment process or after the synchronization establishment, it is desired that a relationship between the light pulse cycle and the reception cycle does not deteriorate.

JP5819387B discloses the above-described advanced ultrasonic imaging system. JP5819387B does not disclose a technology for dealing with the interruption of the detection of the photoacoustic wave. JP2012-29715A discloses a photoacoustic diagnostic apparatus. The photoacoustic diagnostic apparatus does not image a position of the insertion object inserted into the living body.

### SUMMARY OF THE INVENTION

An object of the present disclosure is to provide an ultrasonic imaging system capable of dealing with an interruption in detection of a photoacoustic wave. Alternatively, the object of the present disclosure is to avoid or reduce deterioration in a relationship between a light pulse cycle and a reception cycle in a case where the detection of the photoacoustic wave is interrupted in the ultrasonic imaging system.

An ultrasonic imaging system according to the present disclosure includes a light source that generates a light pulse, an insertion member that has a light absorption element converting the light pulse into a photoacoustic wave and is inserted into a living body, a probe that receives the photoacoustic wave, a determiner that determines detection or non-detection of the photoacoustic wave based on first reception information generated by a reception operation of the probe, a calculator that calculates a synchronization deviation between a light pulse cycle in the light source and a reception cycle in the probe as an actual measurement value based on second reception information generated by the reception operation of the probe, in a case where the detection of the photoacoustic wave is determined, a memory that stores a synchronization deviation history including a plurality of actual measurement values sequentially calculated by the calculator, an estimator that estimates the synchronization deviation as an estimation value based on the synchronization deviation history, in a case where the non-detection of the photoacoustic wave is determined, and a controller that changes at least one cycle of the light pulse cycle or the reception cycle based on the estimation value, in a case where the non-detection of the photoacoustic wave is determined.

A method for controlling synchronization according to the present disclosure includes a step of causing a probe provided to receive a photoacoustic wave to perform a reception operation in a state in which an insertion member having a light absorption element converting a light pulse from a light source into the photoacoustic wave is inserted into a living body, a step of determining detection or non-detection of the photoacoustic wave based on first reception information generated by the reception operation of the probe, a step of calculating a synchronization deviation between a light pulse cycle in the light source and a reception cycle in the probe as an actual measurement value based on second reception information generated by the reception operation of the probe, in a case where the detection of the photoacoustic wave is determined, a step of estimating the synchronization deviation as an estimation value based on a synchronization deviation history including a plurality of actual measurement values sequentially calculated, in a case where the non-detection of the photoacoustic wave is determined, and a step of changing at least one cycle of the light pulse cycle or the reception cycle based on the actual measurement value in a case where the detection of the photoacoustic wave is determined or changing the at least one cycle based on the estimation value in a case where the non-detection of the photoacoustic wave is determined.

According to the present disclosure, deterioration in a relationship between a light pulse cycle and a reception cycle in a case where the detection of the photoacoustic wave is interrupted in the ultrasonic imaging system can be avoided and reduced.

### BRIEF DESCRIPTION OF THE DRAWINGS

FIG. 1 is a block diagram showing an ultrasonic imaging system according to a first embodiment.

FIG. 2 is a block diagram showing a configuration example of a synchronization control device shown in FIG. 1.

FIG. 3 is a diagram showing an example of a synchronization deviation history.

FIG. 4 is a diagram showing an example of a synchronization control.

FIG. 5 is a diagram showing another example of the synchronization control.

FIG. 6 is a flowchart showing a method for controlling synchronization.

FIG. 7 is a timing chart showing a first example of a control for establishing synchronization.

FIG. 8 is a diagram showing an example of a reception signal before phase alignment addition.

FIG. 9 is a diagram showing an example of a reception signal sequence before the phase alignment addition.

FIG. 10 is a diagram showing a spatial relationship between a sound source and a transducer array.

FIG. 11 is a diagram showing a relationship between a synchronization deviation, an actual propagation time, and an apparent propagation time.

FIG. 12 is a diagram showing an example of a method for calculating a synchronization deviation.

FIG. 13 is a timing chart showing a second example of the control for establishing synchronization.

FIG. 14 is a block diagram showing an ultrasonic imaging system according to a second embodiment.

FIG. 15 is a block diagram showing a configuration example of the synchronization control device shown in FIG. 14.

### DESCRIPTION OF THE PREFERRED EMBODIMENTS

Hereinafter, an embodiment will be described based on the drawings.

### (1) Outline of Embodiment

An ultrasonic imaging system according to an embodiment has a light source, an insertion member, a probe, a determiner, a calculator, a memory, an estimator, and a controller. The light source generates a light pulse. The insertion member is a member that has a light absorption element that converts the light pulse into a photoacoustic wave and that is inserted into a living body. The probe receives the photoacoustic wave. The determiner determines detection or non-detection of the photoacoustic wave based on first reception information generated by a reception operation of the probe. In a case where the detection of the photoacoustic wave is determined, the calculator calculates a synchronization deviation between a light pulse cycle in the light source and a reception cycle in the probe as an actual measurement value based on second reception information generated by the reception operation of the probe. A synchronization deviation history including a plurality of actual measurement values sequentially calculated by the calculator is stored in the memory. The estimator estimates the synchronization deviation as an estimation value based on the synchronization deviation history in a case where the non-detection of the photoacoustic wave is determined. The controller changes at least one cycle of the light pulse cycle or the reception cycle based on the estimation value in a case where the non-detection of the photoacoustic wave is determined.

According to the above-described configuration, in a case where the detection of the photoacoustic wave is interrupted, the synchronization deviation is estimated as the estimation value based on the synchronization deviation history. The at least one (target cycle) of the light pulse cycle or the reception cycle is changed based on the estimation value. Therefore, even in a case where the detection of the photoacoustic wave is interrupted, deterioration of a relationship between the light pulse cycle and the reception cycle is avoided or reduced. In general, by executing the above-described processing during a period in which the photoacoustic wave is not detected, a time required for reestablishing a synchronization state after improvement of a situation is shortened.

The estimation value may be stored as a part of the synchronization deviation history, and a new estimation value may be calculated based on the synchronization deviation history including the estimation value. A synchronization control based on the synchronization deviation history may be executed under a situation in which the photoacoustic wave is detected. The reception operation of the probe is an operation executed within a reception period. In a case where the photoacoustic wave is detected, reception information including a photoacoustic wave component is obtained by the reception operation. In a case where the photoacoustic wave is not detected, reception information corresponding to noise is obtained by the reception operation.

In the embodiment, the second reception information is a reception signal sequence consisting of a plurality of reception signals output from a plurality of transducers in the probe or a pseudo-reception signal sequence corresponding to the reception signal sequence. The reception signal sequence is configured of, for example, the plurality of reception signals before a phase alignment addition. For example, the pseudo-reception signal sequence can be regarded as the same as the reception signal sequence from a viewpoint of synchronization deviation calculation. The pseudo-reception signal sequence may be calculated from a beam data sequence, or the pseudo-reception signal sequence may be calculated from image data generated based on the beam data sequence.

In the embodiment, the first reception information is reception information same as the second reception information or reception information different from the second reception information. In general, by making the first reception information and the second reception information the same, the reception information is easily handled.

In the embodiment, the controller switches a method for changing a cycle in a case where a cumulative error condition is satisfied due to continuous determination of the non-detection of the photoacoustic wave. For example, a cumulative error is obtained by calculating an error (cycle correction error) and accumulating the error at each time point, from a time point at which the non-detection of the photoacoustic wave occurs for a first time. The error is obtained, for example, from a magnitude of the estimation value. In a case where the cumulative error is increased, a possibility in which the synchronization control based on the estimation value is inappropriate is increased. Therefore, a method for changing a cycle is switched.

In the embodiment, the controller changes at least one cycle by a first method based on the synchronization deviation history until the cumulative error condition is satisfied in a case where the non-detection of the photoacoustic wave is continuously determined. On the other hand, the controller changes the at least one cycle by a second method different from the first method after the cumulative error condition is satisfied in a case where the non-detection of the photoacoustic wave is continuously determined. For example, the first method is the synchronization control based on the estimation value, and the second method is a synchronization control not based on the estimation value. Examples of the second method include addition of a certain value with respect to the target cycle, subtraction of the certain value from the target cycle, and temporary fixing of the target cycle.

The ultrasound diagnostic apparatus according to the embodiment includes an identifier that identifies a contact state of the probe based on third reception information generated by reception of the photoacoustic wave. The controller changes the at least one cycle based on the contact state of the probe.

In a case where a transmission and reception wave surface of the probe is separated from a surface of the living body, the photoacoustic wave cannot be observed in the probe. In such a state, a necessity to perform a cycle change for synchronization establishment is low. Alternatively, in such a state, appropriate cycle change cannot be performed. Therefore, in the above-described configuration, the contact state is referred to during the synchronization control. The cycle change may be performed only in a case where the contact state is appropriate. The third reception information is the same information as the first reception information or different information from the first reception information, or is the same information as the second reception information or different information from the second reception information. Whether or not the contact state is good may be determined based on an ultrasound image.

A method for controlling synchronization according to the embodiment has a reception step, a determination step, a calculation step, an estimation step, and a control step. In the reception step, in a state in which the insertion member including the light absorption element that converts the light pulse from the light source into the photoacoustic wave is inserted into the living body, the probe provided to receive the photoacoustic wave performs the reception operation. In the determination step, the detection or non-detection of the photoacoustic wave is determined based on the first reception information generated by the reception operation of the probe. In the calculation step, in a case where the detection of the photoacoustic wave is determined, the synchronization deviation between the light pulse cycle in the light source and the reception cycle in the probe is calculated as the actual measurement value based on the second reception information generated by the reception operation of the probe. In the estimation step, in a case where the non-detection of the photoacoustic wave is determined, the synchronization deviation is estimated as the estimation value based on the synchronization deviation history including the plurality of actual measurement values sequentially calculated. In the control step, the at least one cycle of the light pulse cycle or the reception cycle is changed based on the actual measurement value in a case where the detection of the photoacoustic wave is determined, the at least one cycle of the light pulse cycle or the reception cycle is changed based on the estimation value in a case where the non-detection of the photoacoustic wave is determined.

### (2) Details of Embodiment

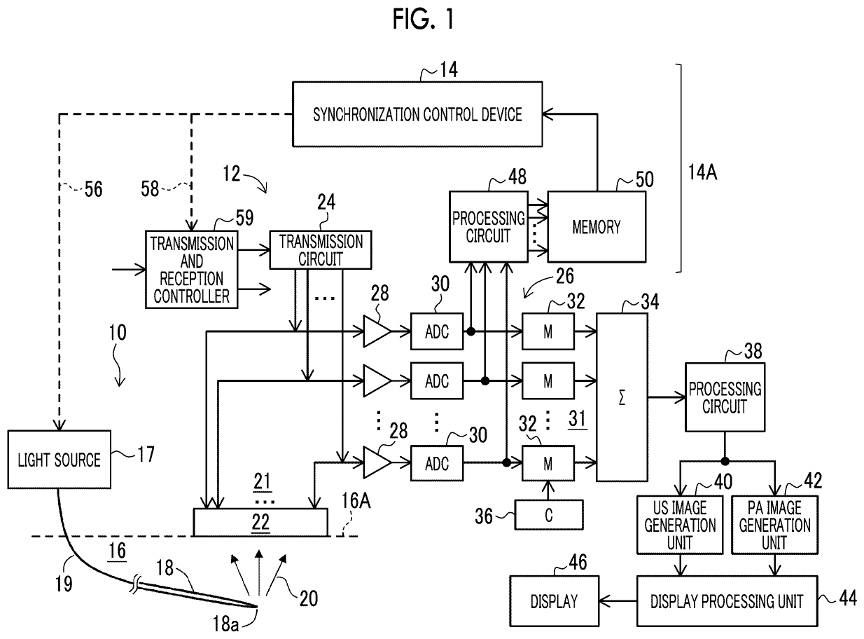

FIG. 1 shows the ultrasonic imaging system according to a first embodiment. The ultrasonic imaging system is a medical system installed in a medical institution such as a hospital, and specifically, is a system used when a distal end of an insertion member is advanced to a lesion while a photoacoustic image (PA image) representing a position of the distal end of the insertion member is observed. The ultrasonic imaging system may be used for other applications. The ultrasonic imaging system includes a light pulse generation device 10, an ultrasound diagnostic apparatus 12, a synchronization control device 14, and an insertion member 18. The light pulse generation device 10 has a light source 17. The light source 17 is a laser that generates a pulse train of laser light. A base end of an optical fiber 19 is connected to the light source 17. A distal end of the optical fiber 19 is provided in a distal end portion of the insertion member 18.

The insertion member 18 is, for example, a catheter inserted into a blood vessel in a living body 16. Other examples of the insertion member 18 include a guide wire and a puncture needle. A light absorption element 18a composed of a light absorption material is provided in the distal end portion of the insertion member 18. The light absorption element 18a absorbs the light pulse by irradiation with the light pulse to the light absorption element 18a. At that time, a photoacoustic wave 20 is generated by a photoacoustic effect. The photoacoustic wave 20 propagates in the living body 16 as a pulse-shaped wave.

Next, the ultrasound diagnostic apparatus 12 as the ultrasound imaging apparatus will be described. An ultrasound probe 21 is in contact with a surface 16A of the living body 16. The ultrasound probe 21 is held by an examiner or a robot. A transducer array 22 configured with the plurality of transducers is provided in the ultrasound probe 21.

In a case where a general tomographic image is formed, the ultrasound wave is transmitted into the living body 16 by the transducer array 22, and a reflection wave from an inside of the living body 16 is received by the transducer array 22. More specifically, a transmission beam and a reception beam are formed and are subjected to electronic scanning.

In a case where the photoacoustic image is formed, the transducer array 22 does not perform a transmission operation and only performs the reception operation. That is, the photoacoustic wave 20 generated in the living body 16 is received by the transducer array 22. More specifically, a plurality of reception periods are set according to the reception cycle on a time axis. Each reception period is a period for receiving or detecting the photoacoustic wave, in other words, corresponds to a reception beam formation period.

In each reception period, a plurality of reception beams may be simultaneously and concurrently formed. All of reception signal sequences obtained from the transducer array 22 may be used as reception information for analysis of the synchronization deviation, or a part of the reception signal sequence may be used as the reception information for the analysis of the synchronization deviation. In general, a transmission and reception process for forming a tomographic image and a reception process for forming a photoacoustic image are alternately executed.

A transmission circuit 24 is a transmission beam former. That is, the transmission circuit 24 is an electronic circuit that concurrently outputs a plurality of transmission signals with respect to the plurality of transducers during transmission.

A reception circuit 26 is a reception beam former corresponding to a receiver or a reception unit. Specifically, the reception circuit 26 is an electronic circuit that processes a plurality of reception signals concurrently output from the plurality of transducers during reception. The reception circuit 26 has a plurality of amplifiers 28 that amplify the plurality of reception signals, an ADC 30 that converts the plurality of reception signals (analog signals) after the amplification, into a plurality of digital signals, and a phase alignment addition unit 31 that applies phase alignment addition to the plurality of reception signals after the conversion. The phase alignment addition is processing of generating reception beam data from the plurality of reception signals.

More specifically, the phase alignment addition unit 31 has a plurality of memories 32 that temporarily store the plurality of reception signals, an adder 34 that adds the plurality of reception signals read out from the plurality of memories, and a controller 36 that controls writing of the plurality of reception signals to the plurality of memories 32 and controls reading out of the plurality of reception signals from the plurality of memories 32. Phases of the plurality of reception signals are aligned by the control of read out timings of the plurality of reception signals from the plurality of memories 32. During the phase alignment addition, so-called reception dynamic focus is performed, and so-called parallel reception is performed as necessary.

The reception circuit 26 outputs the reception beam data generated by the phase alignment addition. By performing electronic scanning of the reception beam once, a plurality of pieces of reception beam data arranged in an electronic scanning direction are generated. By the plurality of pieces of reception beam data, reception frame data corresponding to a beam scanning surface is configured. Each reception beam data is composed of a plurality of pieces of echo data that are arranged in a depth direction. In the reception circuit 26, the reception beam data may be generated by software processing.

A processing circuit 38 is an electronic circuit that processes each reception beam data. The processing circuit 38 includes an envelope detection circuit, a filter circuit, a logarithmic conversion circuit, and the like. During generation of an ultrasound image (US image), each reception beam data output from the processing circuit 38 is transmitted to a US image generation unit 40. During generation of the photoacoustic wave image (PA image), each reception beam data output from the processing circuit 38 is transmitted to a PA image generation unit 42.

The US image generation unit 40 is a module that has a digital scan converter (DSC) and generates display frame data from the reception frame data. Specifically, the US image generation unit 40 generates a tomographic image (B-mode tomographic image) representing a tissue structure as the US image. The DSC has a coordinate transformation function, a pixel interpolation function, and the like.

The PA image generation unit 42 is a module that has the DSC and a sound source specifying unit and generates the display frame data from the reception frame data. The sound source specifying unit specifies a position of the sound source in the living body by detecting or extracting a sound source signal included in the display frame data generated by the DSC. The PA image includes a marker that represents the position of the sound source on a beam scanning surface. The marker is, for example, a point having high brightness or a predetermined color.

A display processing unit 44 generates a composite image by superimposing the PA image on the US image (tomographic image). The composite image is displayed on a display 46. The display 46 is configured with an organic EL display device, a liquid crystal display, or the like.

Each of the US image generation unit 40, the PA image generation unit 42, and the display processing unit 44 is configured by a processor. A CPU that controls an operation of the ultrasound diagnostic apparatus 12 may be allowed to function as the US image generation unit 40, the PA image generation unit 42, and the display processing unit 44.

A transmission and reception controller 59 controls operations of the transmission circuit 24 and the reception circuit 26. The transmission and reception controller 59 determines the reception cycle or a time length of the reception period. The above-described CPU may be allowed to function as the transmission and reception controller 59.

In the ultrasound diagnostic apparatus 12 according to the first embodiment, the reception signal sequence before the phase alignment addition is extracted from the reception circuit 26. The reception signal sequence is configured with a plurality of reception signals output from a plurality of ADCs 30. In a configuration example shown in drawings, the plurality of reception signals extracted are temporarily stored in a memory 50 via a processing circuit 48. Each reception signal read out from the memory 50 is transmitted to the synchronization control device 14. Each reception signal may be output from the processing circuit 48 to the synchronization control device 14. Each reception signal may be directly output from the reception circuit 26 to the synchronization control device 14.

The processing circuit 48 is an electronic circuit that applies necessary signal processing to each reception signal. The necessary signal processing may include envelope detection, noise removal processing, and the like. The processing circuit 48 and the memory 50 may be provided in the synchronization control device 14 (refer to reference numeral 14A).

The synchronization control device 14 is configured with an information processing apparatus, and specifically, is configured with a computer having a CPU that executes a program. The synchronization control device 14 calculates the synchronization deviation between the light pulse cycle and the reception cycle based on the transmitted reception signal sequence, and establishes the synchronization by changing the light pulse cycle or the reception cycle based on the calculated synchronization deviation. Further, in a case where the detection of the photoacoustic wave is interrupted, the synchronization control device 14 estimates the synchronization deviation based on the synchronization deviation history, and maintains the synchronization state or a state close to the synchronization state by changing the light pulse cycle or the reception cycle based on the estimated synchronization deviation, or suppresses an increase in the synchronization deviation.

In a case where the light pulse cycle is changed, a control signal 56 is transmitted from the synchronization control device 14 to the light source 17. The light source 17 changes the light pulse cycle based on the control signal 56. On the other hand, in a case where the reception cycle is changed, a control signal 58 is transmitted from the synchronization control device 14 to the transmission and reception controller 59. The transmission and reception controller 59 changes the reception cycle based on the control signal 58.

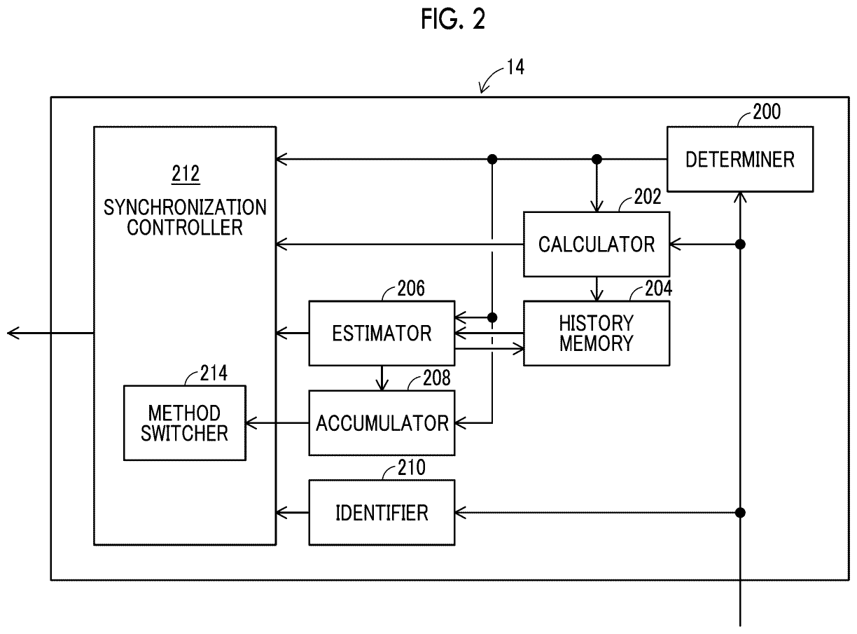

FIG. 2 shows a configuration example of the synchronization control device 14 shown in FIG. 1. The synchronization control device 14 has a CPU and a memory. In FIG. 2, a plurality of functions exhibited by the CPU are represented by a plurality of blocks. Specifically, the synchronization control device 14 has a determiner 200, a calculator 202, an estimator 206, an accumulator 208, an identifier 210, and a synchronization controller 212. Each of the determiner 200, the calculator 202, the estimator 206, the accumulator 208, the identifier 210, and the synchronization controller 212 may be configured with the processor. The synchronization control device 14 also has a history memory 204 configured with a semiconductor memory or the like. The synchronization controller 212 includes a method switcher 214.

The determiner 200 determines the detection or non-detection of the photoacoustic wave based on the reception signal sequence. Specifically, the determiner 200 determines the detection of the photoacoustic wave in a case where a photoacoustic wave signal sequence is included in the reception signal sequence. On the other hand, the determiner 200 determines the non-detection of the photoacoustic wave in a case where the photoacoustic wave signal sequence is not included in the reception signal sequence.

In the configuration example shown in the drawings, a determination result of the determiner 200 is transmitted to the calculator 202, the estimator 206, the accumulator 208, and the synchronization controller 212. The determiner 200 may determine presence or absence of the photoacoustic wave based on other reception information instead of the above-described reception signal sequence. Examples of the other reception information include a pseudo-reception signal sequence described below.

In a case where the detection of the photoacoustic wave is determined, the calculator 202 calculates the synchronization deviation based on the photoacoustic wave signal sequence included in the reception signal sequence. A method for calculating a synchronization deviation will be described in detail below. The calculator 202 may calculate the synchronization deviation based on the other reception information instead of the above-described reception signal sequence. Examples of the other reception information include the pseudo-reception signal sequence described below. In a case where the photoacoustic wave is continuously detected, the calculator 202 sequentially calculates the synchronization deviation. In the following, each synchronization deviation will be represented as an actual measurement value.

A plurality of sequentially calculated actual measurement values are stored in the history memory 204. The synchronization deviation history is configured with the plurality of stored actual measurement values. The history memory 204 is a memory that stores the synchronization deviation history, and is configured of a semiconductor memory or the like. The history memory 204 may store the estimation value of the synchronization deviation, which will be described below.

At a time point of synchronization recovery or at a time point at which a certain period of time has elapsed after the synchronization recovery, stored contents on the history memory 204 may be erased. The stored contents on the history memory 204 may be maintained until an explicit erasure command is input.

In a case where the non-detection of the photoacoustic wave is determined, the estimator 206 estimates a current synchronization deviation as an estimation value based on the synchronization deviation history, that is, based on a series of past synchronization deviations. For example, the estimation value may be calculated by averaging the plurality of actual measurement values in a past certain period. A weighted averaging method or an extrapolation method may be adopted instead of a simple averaging method. Examples of a filter applied to the synchronization deviation history for estimating the synchronization deviation include a simple averaging filter, a low-pass filter, and a most frequent value filter. A reference section on the synchronization deviation history may be adaptively changed.

The accumulator 208 accumulates the calculated error to obtain the cumulative error within a period in which the non-detection of the photoacoustic wave is continuously determined. For example, the error is calculated by multiplying the estimation value by a certain coefficient. The certain coefficient is, for example, 0.2. The cumulative error is transmitted to the method switcher 214.

The identifier 210 identifies the contact state of the probe based on the reception signal sequence, and specifically, determines a suitability of the contact state. For example, in a case where the transmission and reception wave surface of the probe is separated from the surface of the living body, overall amplitude of each reception signal is greatly reduced. In a case where a part of the transmission and reception wave surface is detached from the surface of the living body, an amplitude of a part of the reception signal is also reduced. The suitability of the contact state of the probe is determined from such an amplitude change. The identifier 210 may identify the contact state based on other reception information instead of the above-described reception signal sequence. Examples of the other reception information include a pseudo-reception signal sequence described below. The contact state may be identified based on the ultrasound image.

In a case where the detection of the photoacoustic wave is determined, the synchronization controller 212 performs the synchronization control based on the actual measurement value. Specifically, at least one (target cycle) of the light pulse cycle in the light source or the reception cycle in the probe is changed based on the actual measurement value. As a result, it is possible to synchronize the light pulse cycle and the reception cycle with high accuracy.

In the beginning of the synchronization control, in at least some cases, the photoacoustic wave is not detected and the synchronization deviation history is also not generated. In such a situation, the synchronization controller 212 changes the target cycle by adding a certain value to the target cycle or subtracting the certain value from the target cycle. By repeating such a change in the target cycle, the photoacoustic wave is detected, and sequentially synchronization is established.

In a case where the determination of the non-detection of the photoacoustic wave is repeated and the cumulative error exceeds a threshold value, the method switcher 214 switches the method for controlling synchronization. Specifically, the cycle change based on the estimation value is switched to the cycle change using the certain value. In that case, for example, a new target cycle is calculated and set by adding the certain value to the target cycle or subtracting the certain value from the target cycle.

In a case where the identifier 210 determines a defect of the contact state, the synchronization controller 212 temporarily suspends the cycle change. That is, a last cycle before the determination of the defect of the contact state is maintained. In a case where the appropriate contact state is recovered, the synchronization control, that is, the cycle change is resumed. In that case, for example, the cycle change based on the actual measurement value may be performed after the cycle change by the addition or subtraction of the certain value is performed.

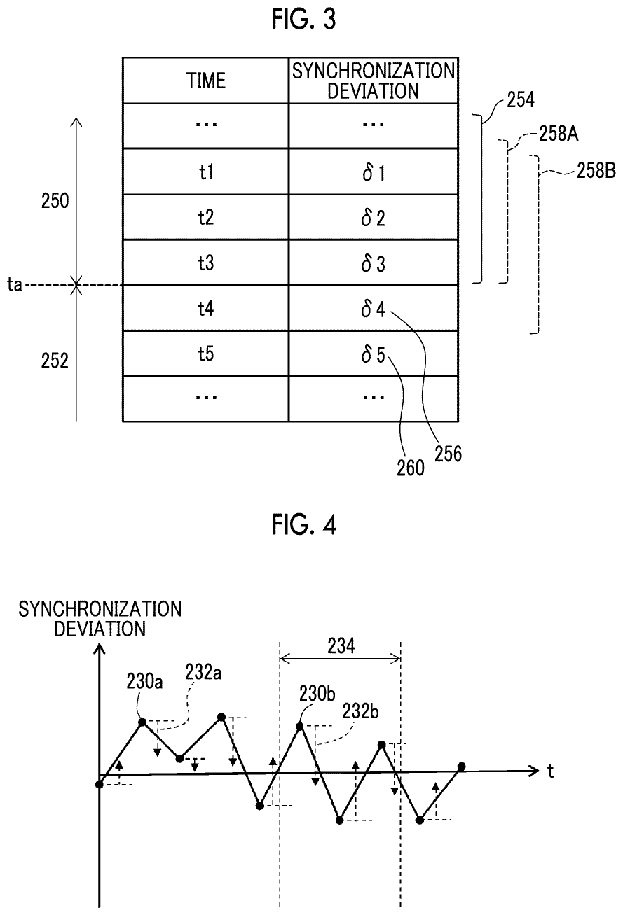

FIG. 3 shows an example of the synchronization deviation history. The synchronization deviation history is configured of a plurality of synchronization deviations corresponding to a plurality of time points. A time point ta indicates a timing at which the detection of the photoacoustic wave is interrupted. A period 250 is a period in which the determination of the detection of the photoacoustic wave is repeated, and a period 252 is a period in which the determination of the non-detection of the photoacoustic wave is repeated.

For example, a synchronization deviation (estimation value) 256 at a time point t4 is estimated based on a plurality of synchronization deviations (plurality of actual measurement values) belonging to a period 254. Next, for example, a synchronization deviation (estimation value) 260 at a time point t5 is calculated based on a plurality of synchronization deviations (plurality of actual measurement values) belonging to a period 258A. The synchronization deviation (estimation value) 260 at the time point t5 may be calculated based on a plurality of synchronization deviations (plurality of actual measurement values and one estimation value) belonging to a period 258B. In a case where such a calculation is performed, each estimation value calculated is stored in the history memory. Within the period 252, the synchronization deviation is repeatedly estimated based on the synchronization deviation history. However, the estimation of the synchronization deviation is stopped at a time point at which the cumulative error condition is satisfied.

FIG. 4 shows an example of the synchronization control. A horizontal axis is a time axis, and a vertical axis indicates a magnitude of the synchronization deviation. Each point represents the synchronization deviation (actual measurement value or estimation value) calculated at each time point. A period 234 is a non-detection period of the photoacoustic wave. FIG. 4 is for describing the synchronization control according to the embodiment in an easily understandable manner and the contents thereof are exaggerated or different from actual ones.

For example, the target cycle is changed based on an actual measurement value 230a of the synchronization deviation such that the synchronization deviation is reduced or the synchronization deviation is resolved (refer to a reference numeral 232a). Such processing is repeated. In a case where the detection of the photoacoustic wave is interrupted, that is, within the non-detection period 234, an estimation value 230b is calculated based on the synchronization deviation history, and the target cycle is changed based on an estimation value 230c (refer to a reference numeral 232b). Such processing is repeated within the non-detection period 234. After the non-detection period 234 elapses, that is, in a case where the detection of the photoacoustic wave is resumed, the change of the target cycle based on the actual measurement value is resumed.

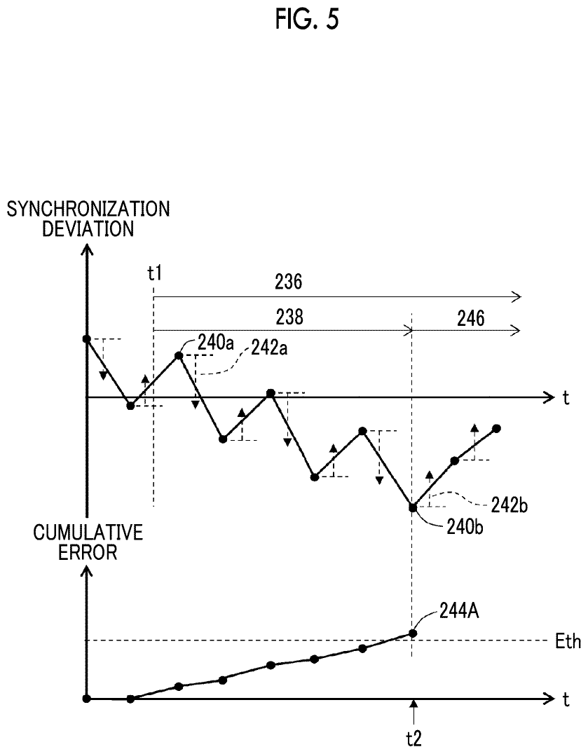

FIG. 5 shows another example of the synchronization control. In an upper part, a horizontal axis is a time axis, and a vertical axis indicates the magnitude of the synchronization deviation. In a lower part, a horizontal axis is a time axis, and a vertical axis indicates a magnitude of the cumulative error. FIG. 5 is for describing the synchronization control according to the embodiment in an easily understandable manner and the contents thereof are exaggerated or different from actual ones.

The detection of the photoacoustic wave is interrupted at a time point t1. A period 236 is a non-detection period of the photoacoustic wave. In a period 238 in the period 236, the cycle change based on the estimation value, that is, the first method is performed. Specifically, an estimation value 240a is calculated based on the synchronization deviation history, and the target cycle is changed based on the estimation value 240a (reference numeral 242a). The above-described processing is repeated. The error is calculated by multiplying each estimation value to be calculated by the certain coefficient, and the cumulative error is calculated by accumulating each error. The cumulative error gradually increases within the period 238.

At a time point t2, a cumulative error 244A exceeds a threshold value Eth, that is, the cumulative error condition is satisfied. Accordingly, the method for changing a cycle is switched from the first method to the second method. A period 246 is a period in which the second method is performed. The second method is a method for determining a new target cycle by, for example, adding the certain value to the target cycle or subtracting the certain value from the target cycle. Under the new target cycle, a synchronization deviation 240b occurs. The cycle change based on the second method is repeated within the period 246. A method other than the above may be adopted as a second modification method. Instead of the cumulative error, an elapsed time from the time point at which the detection of the photoacoustic wave is interrupted may be referred to.

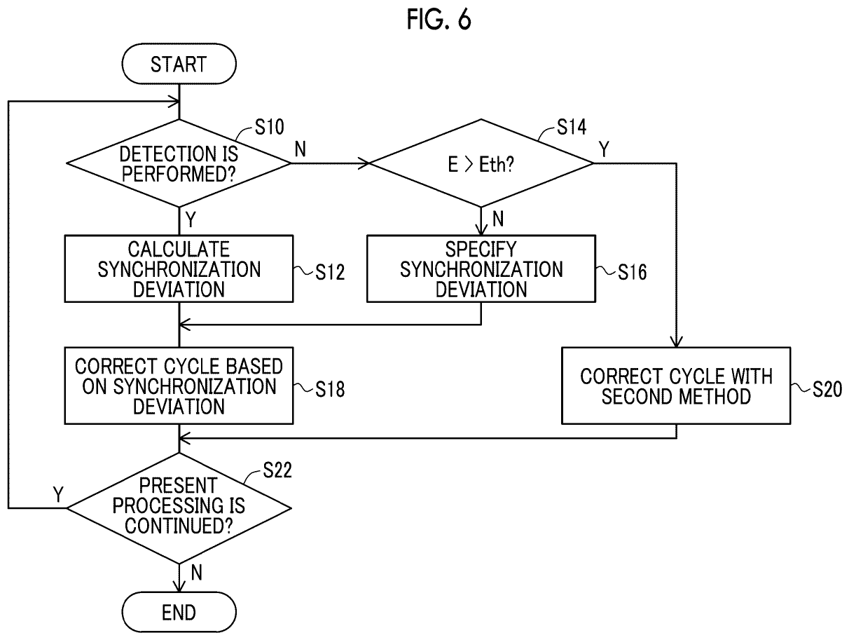

FIG. 6 shows the synchronization control that is executed after a certain number of the actual measurement values are acquired (that is, in a transition process before the synchronization establishment or after the synchronization establishment). In S10, the detection or non-detection of the photoacoustic wave is determined. In a case where the detection is determined, the synchronization deviation (actual measurement value) is calculated based on the photoacoustic wave signal sequence included in the reception signal sequence based on S12.

On the other hand, in a case where the non-detection is determined in S10, it is determined whether or not a cumulative error E exceeds the threshold value Eth in S14. In a case where the cumulative error E does not exceed the threshold value Eth, the first method is performed in S16. That is, the synchronization deviation (estimation value) is calculated based on the synchronization deviation history. In S18, the target cycle is corrected based on the synchronization deviation (the actual measurement value or the estimation value).

In S14, in a case where it is determined that the cumulative error E exceeds the threshold value Eth, in S20, the second method different from the first method is performed, that is, the target cycle is corrected by the second method. In S22, it is determined whether or not to continue the present processing, and in a case of continuing the present processing, each step after S10 is executed again.

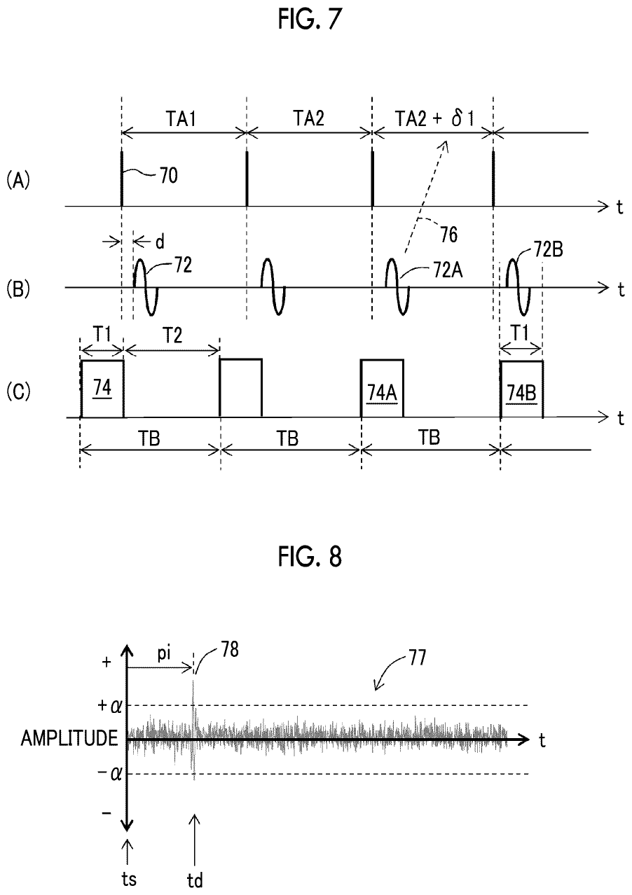

Hereinafter, the method for calculating a synchronization deviation will be described using FIGS. 7 to 13. FIG. 7 shows a first example of the control for establishing the synchronization as a timing chart. (A) of FIG. 7 shows a light pulse train generated by the light source. The light pulse train is configured by a plurality of light pulses 70 arranged on the time axis. A width of the light pulse is, for example, 100 ns, and the light pulse cycle is, for example, 1 ms.

(B) of FIG. 7 shows a plurality of photoacoustic wave signal sequences 72 arranged on the time axis. In FIG. 7, each photoacoustic wave signal sequence 72 is schematically represented, that is, represented as a single pulse wave. A plurality of photoacoustic wave signals are generated concurrently by the plurality of transducers receiving a photoacoustic wave generated by one light pulse. The photoacoustic wave signal sequence 72 is configured with the plurality of photoacoustic wave signals. d indicates a propagation time of the photoacoustic wave.

(C) of FIG. 7 shows a plurality of reception periods 74 arranged on the time axis. Each reception period 74 is a period in which the photoacoustic wave can be received. Each reception period 74 is set in the ultrasound probe, in other words, is set for the reception circuit. For example, a reception opening is set for an entire transducer array, and a reception signal sequence corresponding to the reception opening is analyzed.

In the first example shown in FIG. 7, the light pulse cycle is changed in the light pulse cycle and the reception cycle based on the calculated synchronization deviation. Each of TA1, TA2, and TA2+δ1 indicates a light pulse cycle. TB indicates a reception cycle. A reception cycle TB is fixed. A time length of each of the reception period 74 is T1. Incidentally, T2 is a time length of a blank period.

In a case where the photoacoustic wave signal sequence 72 cannot be observed in the reception period 74, the light pulse cycle is changed on a trial basis. Specifically, a current light pulse cycle TA2 is set by adding a certain period to a previous light pulse cycle TA1 or subtracting the certain period from the light pulse cycle TA1.

In the example shown in FIG. 7, a photoacoustic wave signal sequence 72A is observed in a reception period 74A. A synchronization deviation 81 is calculated by analyzing the photoacoustic wave signal sequence 72A (refer to reference numeral 76). For example, by adding the synchronization deviation 81 to the current light pulse cycle TA2, a new light pulse cycle TA2+δ1 is set. As a result, synchronization between the light pulse cycle and the reception cycle is established. That is, the magnitude of the light pulse cycle and the magnitude of the reception cycle are the same, and a photoacoustic wave signal sequence 72B is correctly observed in a reception period 74B. Thereafter, as necessary, in order to maintain a synchronization establishment state, the calculation of the synchronization deviation and the control based on the synchronization deviation are continuously performed. In order to establish the synchronization, a generation timing of the light pulse and a start timing of the reception period may be matched.

FIG. 8 shows an example of the reception signal before the phase alignment addition. A reception signal 77 includes a photoacoustic wave signal 78 generated due to the reception of the photoacoustic wave. The photoacoustic wave signal 78 has a form with a peak shape or a pulse shape having a large amplitude. For example, the photoacoustic wave signal 78 may be detected or extracted by threshold value processing. In this case, a portion exceeding a threshold value a is specified as the photoacoustic wave signal 78. The threshold value a may be set according to the magnitude of a noise included in the reception signal. For example, in a case where a standard deviation of the reception signal is represented by σ, the threshold value a may be set in accordance with α=6σ. Before the threshold value processing, filter processing, envelope detection, or the like may be applied to the reception signal.

The detection of the photoacoustic wave signal 78 specifies a timing (detection timing) td at which the photoacoustic wave is detected for each reception signal. A period pi from a reception period start timing ts to the detection timing td includes a time corresponding to the propagation time of the photoacoustic wave and the synchronization deviation. From such a viewpoint, in the following, the period pi will be referred to as an apparent propagation time.

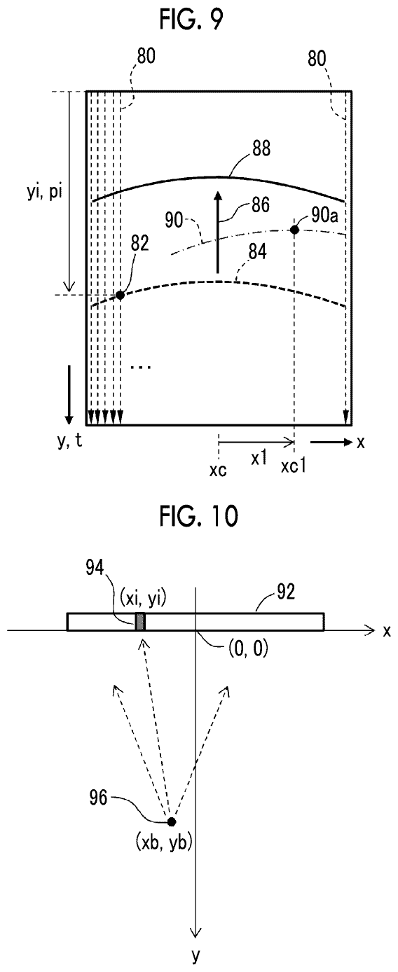

FIG. 9 shows an example of the reception signal sequence before the phase alignment addition. The reception signal sequence is configured by a plurality of reception signals 80 corresponding to the plurality of transducers constituting the transducer array. In FIG. 9, the x direction is a transducer arrangement direction, and the y direction is a depth direction. The y direction corresponds to the time axis.

A photoacoustic wave signal sequence 84 is included in the reception signal sequence. In the example shown in the drawing, an x-coordinate (xc) of a center of a transducer array matches an x-coordinate of the sound source, and an x-coordinate of a vertex of the photoacoustic wave signal sequence 84 matches an x-coordinate (xc) of a center of the transducer array.

The photoacoustic wave signal sequence 84 is composed of the plurality of photoacoustic wave signals. In a case where a specific photoacoustic wave signal 82 received by an i-th transducer is focused on, the apparent propagation time pi is specified based on the detection timing. The specific photoacoustic wave signal 82 is generated at a point at a depth yi.

The photoacoustic wave signal sequence 84 has a parabolic form. The form of the photoacoustic wave signal sequence 84 is constant regardless of the magnitude of the synchronization deviation. A position at which the photoacoustic wave signal sequence 84 is generated changes depending on a spatial relationship between the transducer array and the sound source. For example, in a case where the position of the sound source is shifted in the x direction from a center position xc of the transducer array, a photoacoustic wave signal sequence 90 is generated. An x-coordinate (xc1) of a vertex 90a corresponds to an x-coordinate of the sound source. Even in that case, a form of the photoacoustic wave signal sequence 90 is the same as the form of the photoacoustic wave signal sequence 84.

The synchronization deviation is calculated based on the photoacoustic wave signal sequence 84, and at least one of the light pulse cycle or the reception cycle is changed based on the synchronization deviation. Accordingly, the photoacoustic wave signal sequence 84 is moved parallel to the depth direction in a coordinate space shown in FIG. 9 (refer to reference numeral 86). A reference numeral 88 represents a photoacoustic wave signal sequence to be observed in a case where the synchronization deviation is resolved.

Hereinafter, the method for calculating a synchronization deviation will be described in more detail. FIG. 10 shows a spatial relationship between a transducer array 92 and a sound source 96. The x direction is a transducer arrangement direction, and the y direction is a depth direction. A center of the transducer array 92 is an origin point (0, 0), and a position of an i-th transducer 94 is represented by (xi, yi). Here, yi=0. A position of the sound source 96 is represented by (xb, yb). A propagation time di of the photoacoustic wave from the sound source 96 to the i-th transducer is calculated by Expression (1). (1) c in the expression is an acoustic velocity of an ultrasound wave (photoacoustic wave) in a medium.

$${d}_{i} = \frac{\sqrt{{( {x}_{i} - {x}_{b} )}^{2} + {y}_{b}^{2}}}{c} \qquad ( 1 )$$

The apparent propagation time pi is a time obtained by adding the synchronization deviation δ and the propagation time di. That is, the apparent propagation time pi is represented by Expression (2).

$${p}_{i} = δ + \frac{\sqrt{{( {x}_{i} - {x}_{b} )}^{2} + {y}_{b}^{2}}}{c} \qquad ( 2 )$$

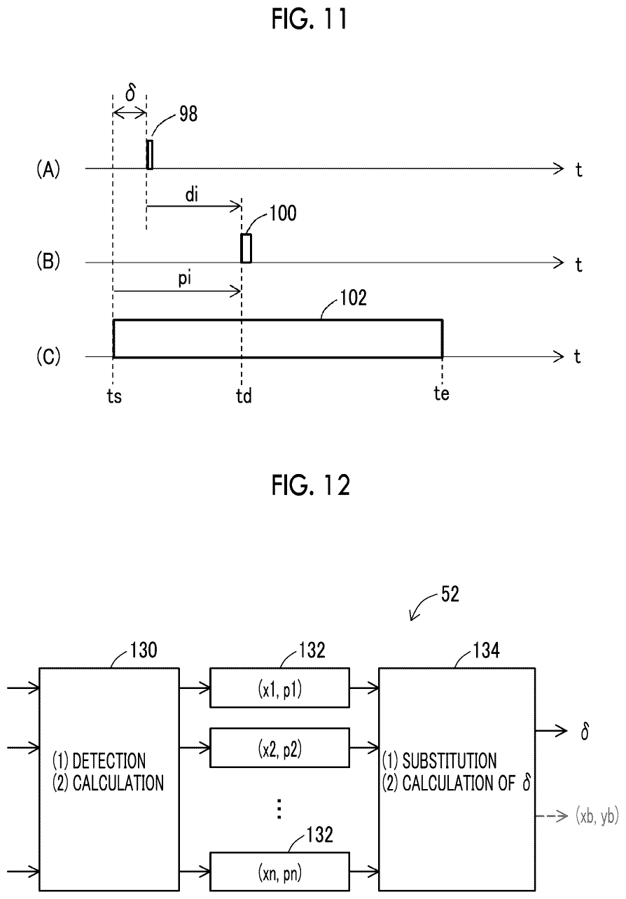

A relationship among the apparent propagation time pi, the synchronization deviation δ, and the propagation time (actual propagation time) di, which are represented by Expression (2), is shown in FIG. 11. A reception period 102 is a period from the start timing ts to an end timing te. A time between the start timing ts and a generation timing of a light pulse 98 is the synchronization deviation δ. A propagation time from a generation timing (generation timing of the photoacoustic wave) of the light pulse 98 to reception of the photoacoustic wave by the i-th transducer is di. Reference numeral 100 represents the photoacoustic wave signal generated by receiving the photoacoustic wave.

The photoacoustic wave reaches n transducers, and n detection timings td corresponding to the n transducers are specified. n apparent propagation times pi are specified based on n detection timings td. n data pairs (xi, pi) are defined by positions xi of the n transducers and the n apparent propagation times pi (where, i=1, . . . , n).

By substituting n data pairs (xi, pi) into Expression (2) which is a mathematical model, δ, xb, and yb which are unknown parameters can be specified. In this case, a solution search method such as a least squares method is used. In this method, in addition to the synchronization deviation δ, coordinates (xb, yb) of the sound source are also specified.

FIG. 12 schematically shows the method described above. In a block 130, the n photoacoustic wave signals included in the n reception signals before the phase alignment addition are detected. In addition, the n apparent propagation times pi from the reception period start timing to the n detection timings are calculated. Coordinates xi of n transducers in the x direction and the n apparent propagation times pi define n data pairs (xi, pi) 132. In a block 134, the synchronization deviation δ, which is the unknown parameter, is calculated by substituting the n data pairs (xi, pi) 132 into Expression (2). The coordinates (xb, yb) of the sound source are also calculated as a secondary effect. Among the n data pairs (xi, pi) 132, only a plurality of data pairs (xi, pi) 122 satisfying a certain condition may be substituted into Expression (2).

As described above, the synchronization deviation δ is calculated by analyzing the plurality of reception signals. At least one of the light pulse cycle or the reception cycle is changed based on the synchronization deviation. In a case where the light pulse cycle is changed, the reception cycle can be maintained, and thus, it is possible to obtain an advantage that it is not necessary to change a transmission and reception sequence in the ultrasound diagnostic apparatus. In a case where the reception cycle is changed, communication between the information processing apparatus (or the ultrasound diagnostic apparatus) and the light pulse generation device is not required. Therefore, a configuration of the ultrasonic imaging system can be simplified.

In a case where |xi−xb| in Expression (2) is focused on, |xi−xb|<<yb is satisfied in many cases. Therefore, the following Expression (3) is satisfied for Expression (2).

$${p}_{i} = δ + \frac{{y}_{b}}{c} \sqrt{1 + {( \frac{{x}_{i} - {x}_{b}}{{y}_{b}} )}^{2}} ≃ δ + \frac{{y}_{b}}{c} + \frac{1}{2 {y}_{b} c} {( {x}_{i} - {x}_{b} )}^{2} \qquad ( 3 )$$

Expression (3) indicates that the plurality of photoacoustic wave signals draw a parabola in the above-described xy coordinate system. The synchronization deviation & may be specified using Expression (3). The photoacoustic image may be generated by using the coordinates (xb, yb) of the specified sound source, or an operation of the ultrasonic imaging system may be controlled based on the coordinates (xb, yb) of the specified sound source.

Incidentally, in the synchronization control, the smoothed synchronization deviation may be referred to as the actual measurement value instead of directly referring to the synchronization deviation calculated at each time point. In this case, for example, the smoothed synchronization deviation may be calculated according to Expression (4) below, and a next cycle PRTⱼ₊₁ may be determined based on the calculated smoothed synchronization deviation.

$${PRT}_{j \to j + 1} = PRT + \frac{1}{N} {\sum }_{0}^{N - 1} {δ}_{j} \qquad ( 4 )$$

In Expression (4), δⱼ represents j-th synchronization deviation. PRTⱼ₊₁ indicates a (j+1)th light pulse cycle calculated based on a j-th light pulse cycle. In Expression (4), N synchronization deviation amounts are averaged, and an average value of the synchronization deviation amounts is added to the j-th light pulse cycle. The smoothed light pulse cycle may be calculated based on another calculation expression.

The certain coefficient (for example, 0.8) may be multiplied by the calculated synchronization deviation or the smoothed synchronization deviation, and the light pulse cycle or the reception cycle may be corrected based on the actual measurement value of the synchronization deviation obtained by the multiplication.

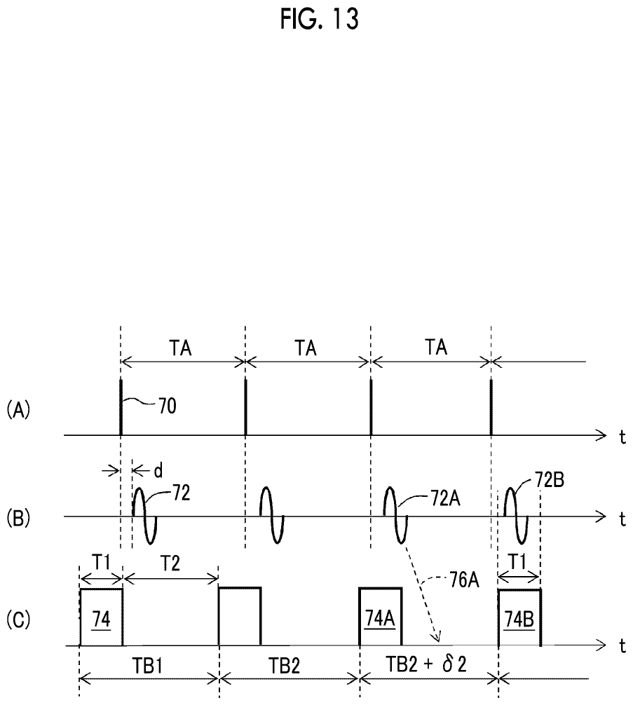

FIG. 13 shows a second example of the control for establishing the synchronization as a timing chart. In FIG. 13, the same elements as the elements shown in FIG. 7 are denoted by the same reference numerals, and the description thereof will not be shown.

In FIG. 13, each of TB1, TB2, and TB2+δ2 indicates the reception cycle. The light pulse cycle TA is fixed. In a case where the photoacoustic wave signal sequence 72 is not detected in the reception period 74, the reception cycle is changed on a trial basis. Specifically, for example, a next reception cycle TB2 is set by adding the certain period to a reception cycle TB1.

In the reception period 74A, the photoacoustic wave signal sequence 72A is observed. Synchronization deviation δ2 is calculated by analyzing the photoacoustic wave signal sequence 72A (refer to reference numeral 76A), and in the example shown in the drawing, a new reception cycle TB2+δ2 is set by adding the synchronization deviation δ2 to the current reception cycle TB2. As a result, synchronization between the light pulse cycle and the reception cycle is established. Specifically, the photoacoustic wave signal sequence 72B is observed in the reception period 74B.

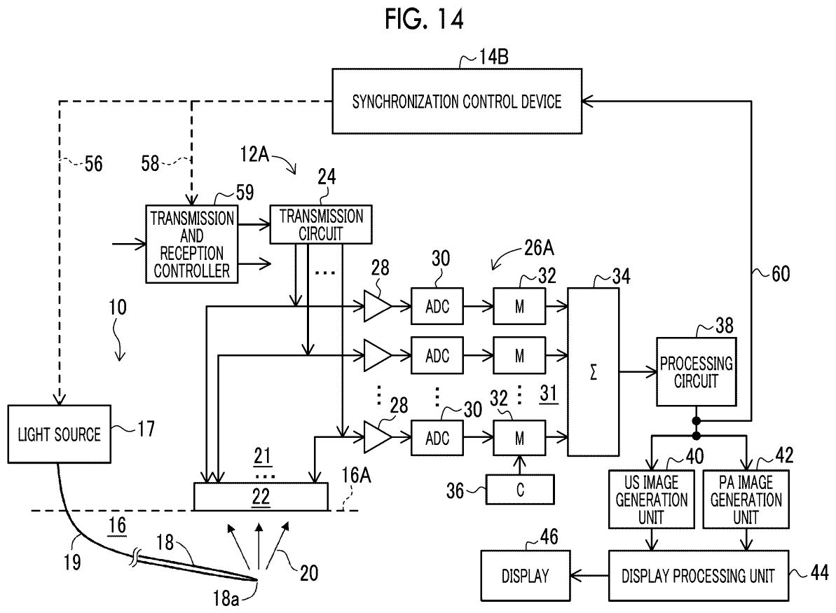

FIG. 14 shows an ultrasonic imaging system according to a second embodiment. In FIG. 14, the same elements as the elements shown in FIG. 1 are denoted by the same reference numerals, and the description thereof will not be shown.

In the second embodiment, reception information 60 output from the processing circuit 38 is transmitted to an information processing apparatus 14B. The reception information 60 is a plurality of pieces of reception beam data that have been subjected to certain processing. Reception information output from a reception circuit 26A may be transmitted to the information processing apparatus 14B. A PA image to be described later may be transmitted to the information processing apparatus 14B as the reception information. By transmitting the plurality of pieces of reception beam data after the envelope detection, an amount of transmission data can be significantly reduced.

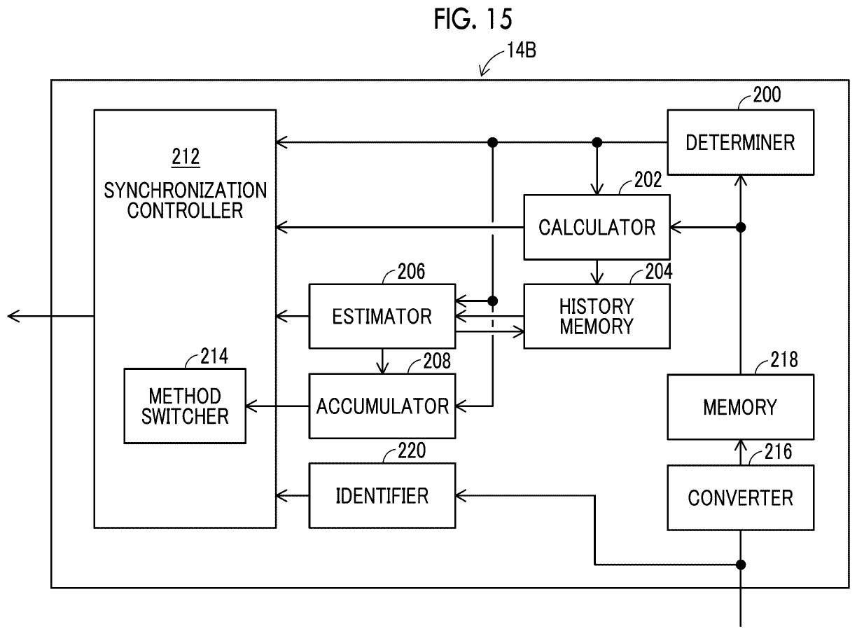

FIG. 15 shows a configuration example of the information processing apparatus 14B. In FIG. 15, the same elements as the elements shown in FIG. 2 are denoted by the same reference numerals, and the description thereof will not be shown.

A converter 216 applies a calculation inverse to the phase alignment addition with respect to the plurality of pieces of beam data transmitted. The inverse calculation is also referred to as an inverse Fourier transform or an inverse phase alignment addition. The converter 216 is a restorer in view of the above point. The pseudo-reception signal sequence corresponding to the reception signal sequence before the phase alignment addition is output from the converter 216. The pseudo-reception signal sequence is temporarily stored in a memory 218.

In a case where the pseudo-reception signal sequence is mapped to an xy-coordinate space, the same photoacoustic wave signal sequence as the photoacoustic wave signal sequence shown in FIG. 9 is generated. In FIG. 15, the determiner 200 determines the detection or non-detection of the photoacoustic wave based on whether or not the photoacoustic wave signal sequence is included in the pseudo-reception signal sequence. The calculator 202 calculates the synchronization deviation based on the photoacoustic wave signal sequence included in the pseudo-reception signal sequence as the actual measurement value. An identifier 220 identifies the contact state of the probe based on the reception information (the plurality of pieces of beam data) transmitted from the ultrasound diagnostic apparatus. That is, it is determined whether or not the contact state is good.

Other reception information instead of the pseudo-reception signal sequence may be input to the determiner 200. For example, the PA image may be input. In this case, the detection or non-detection of the photoacoustic wave may be determined based on brightness information in the PA image, specifically, based on the presence or absence of an image or a pixel group having a brightness value exceeding a certain threshold value. Other reception information instead of the pseudo-reception signal sequence may be input to the identifier 220. For example, the PA image may be input in the same manner as described above. In that case, based on the brightness information in the PA image, for example, based on an average brightness, whether or not the contact state of the probe is good may be determined.

According to the second embodiment, an advantage is obtained, in that transmission information is easily extracted from the ultrasound diagnostic apparatus. All or a part of a constituent element group shown in FIG. 15 may be provided in the ultrasound diagnostic apparatus. Alternatively, all or a part of the constituent element group shown in FIG. 15 may be provided in the light pulse generation device.

According to the ultrasonic imaging systems according to the first embodiment and the second embodiment, in a case where the detection of the photoacoustic wave is interrupted, the synchronization deviation is estimated as an estimation value based on the synchronization deviation history. At least one cycle of the light pulse cycle or the reception cycle is changed based on the estimation value. Therefore, even in a case where the detection of the photoacoustic wave is interrupted, the synchronization state or a state close to the synchronization state is maintained. By continuously optimizing the target cycle during the period in which the photoacoustic wave is not detected, the time required for reestablishing the synchronization state after the improvement of the situation is shortened.

What is claimed is:

1. An ultrasonic imaging system comprising:
a light source that generates a light pulse;
an insertion member that has a light absorption element converting the light pulse into a photoacoustic wave and is inserted into a living body;
a probe that receives the photoacoustic wave;
a determiner that determines detection or non-detection of the photoacoustic wave based on first reception information generated by a reception operation of the probe;
a calculator that calculates a synchronization deviation between a light pulse cycle in the light source and a reception cycle in the probe as an actual measurement value based on second reception information generated by the reception operation of the probe, in a case where the detection of the photoacoustic wave is determined;
a memory that stores a synchronization deviation history including a plurality of actual measurement values sequentially calculated by the calculator;
an estimator that estimates the synchronization deviation as an estimation value based on the synchronization deviation history, in a case where the non-detection of the photoacoustic wave is determined; and
a controller that changes at least one cycle of the light pulse cycle or the reception cycle based on the estimation value, in a case where the non-detection of the photoacoustic wave is determined.

2. The ultrasonic imaging system according to claim 1,
wherein the second reception information is a reception signal sequence consisting of a plurality of reception signals output from a plurality of transducers in the probe or a pseudo-reception signal sequence corresponding to the reception signal sequence.

3. The ultrasonic imaging system according to claim 2,
wherein the first reception information is reception information same as the second reception information or reception information different from the second reception information.

4. The ultrasonic imaging system according to claim 1,
wherein the controller switches a method for changing the at least one cycle, in a case where a cumulative error condition is satisfied due to continuous determination of the non-detection of the photoacoustic wave.

5. The ultrasonic imaging system according to claim 4,
wherein the controller
changes the at least one cycle by a first method based on the synchronization deviation history until the cumulative error condition is satisfied in a case where the non-detection of the photoacoustic wave is continuously determined, and
changes the at least one cycle by a second method different from the first method after the cumulative error condition is satisfied in a case where the non-detection of the photoacoustic wave is continuously determined.

6. The ultrasonic imaging system according to claim 1, further comprising:
an identifier that identifies a contact state of the probe based on third reception information generated by the reception operation of the probe,
wherein the controller changes the at least one cycle based on the contact state of the probe.

7. A method for controlling synchronization, the method comprising:
a step of causing a probe provided to receive a photoacoustic wave to perform a reception operation in a state in which an insertion member having a light absorption element converting a light pulse from a light source into the photoacoustic wave is inserted into a living body;
a step of determining detection or non-detection of the photoacoustic wave based on first reception information generated by the reception operation of the probe;
a step of calculating a synchronization deviation between a light pulse cycle in the light source and a reception cycle in the probe as an actual measurement value based on second reception information generated by the reception operation of the probe, in a case where the detection of the photoacoustic wave is determined;
a step of estimating the synchronization deviation as an estimation value based on a synchronization deviation history including a plurality of actual measurement values sequentially calculated, in a case where the non-detection of the photoacoustic wave is determined; and
a step of changing at least one cycle of the light pulse cycle or the reception cycle based on the actual measurement value in a case where the detection of the photoacoustic wave is determined or changing the at least one cycle based on the estimation value in a case where the non-detection of the photoacoustic wave is determined.
